(19) United States
(12) Patent Application Publication
(10) Pub. No.: US 2022/0280135 A1
(43) Pub. Date: Sep. 8, 2022

(54) ENDOSCOPE OR CATHETER ASSEMBLIES INCLUDING TWO OR MORE EXIT PORTS

(71) Applicant: Covidien LP, Mansfield, MA (US)

(72) Inventors: Paul M. Galluzzo, Godmanchester (GB); Rita Stella, Sandy (GB); Neil Pollock, Wimpole (GB)

(21) Appl. No.: 17/665,444

(22) Filed: Feb. 4, 2022

Related U.S. Application Data
(60) Provisional application No. 63/156,894, filed on Mar. 4, 2021.

(51) Int. Cl. A61B 8/00; A61B 1/018; A61B 1/267; A61B 8/12; A61B 1/005

(52) U.S. Cl. CPC A61B 8/445; A61B 1/018; A61B 1/2676; A61B 8/12; A61B 1/005; A61B 8/4254

(57) ABSTRACT

The present disclosure describes endoscope assemblies or catheter assemblies including two or more exits ports and navigation systems associated therewith.

### CROSS-REFERENCE TO RELATED APPLICATIONS

This application claims benefit of and priority to U.S. Provisional Patent Application Nos. 63/156,894 filed Mar. 4, 2021, the disclosure of which is hereby incorporated by reference in its entirety.

### BACKGROUND

### Technical Field

The present technology is generally related to endoscopes or catheters including two or more exit ports, and more particularly, endoscope or catheter assemblies designed for navigation within a luminal body structure and including two or more exit ports.

### Description of Related Art

A wide variety of endoscopes and catheters, as well as surgical instruments designed to be used with such devices, have been developed. Of these known devices, each has certain advantages and disadvantages. However, there is an ongoing need to provide alternative endoscopes and/or catheters. For example, in some instances, some known endoscopes and/or catheters may be unable to properly articulate inside a given tissue lumen and/or device channel thereby preventing proper alignment of the surgical instrument and/or catheter to the target tissue. Particularly, articulation of a catheter or surgical instrument may cause a distal portion of an endoscope or catheter, respectively, to shift from an aligned position to an unaligned position with a target tissue. Thus, there exists a need to provide endoscopes and/or catheters having an ability to more efficiently align a catheter and/or surgical instrument next to a target tissue.

### SUMMARY

The present disclosure describes endoscope assemblies and/or catheter assemblies including at least two exits ports, and particularly at least one exit port on a distal end of the assembly and at least one compound exit port positioned on a distal end portion of the assembly proximal to the distal end port.

In some embodiments, the present disclosure describes an endoscope assembly configured for navigation within a luminal structure, the endoscope assembly includes an endoscope, a catheter, and optionally a surgical instrument. The endoscope has a shaft portion including an endoscope sidewall that defines an endoscope channel therein, a distal endoscope port positioned on a distal end of the shaft portion and in communication with a distal end of the endoscope channel, and an angled side endoscope port defined through a distal portion of the endoscope sidewall and in communication with the endoscope channel. The catheter is configured for positioning within the endoscope channel of the endoscope. The catheter is also configured to extend distally through the distal endoscope port or extend laterally through the angled side endoscope port. In some instances, the catheter may be configured to be curved. In some instances, the catheter may be pre-curved or include a fix curved, in particular an elliptical fixed curve. In some instances, the catheter may be steerable or articulatable to form a curve.

The angled side endoscope port is a compound opening. For example, the angled side endoscope port includes two or more edges to define the port or opening. In some instances, the compound opening defines at least a first edge along the endoscope sidewall and extending generally perpendicular to a longitudinal axis of the endoscope channel and a second edge along the endoscope sidewall and extending at an acute angle relative to the longitudinal axis of the endoscope channel. In some instances, the first and second edges form a first angle therebetween ranging from about 25° to about 85° or from about 35° to about 75°.

In some instances, the endoscopes described herein are bronchoscopes. In some instances, the endoscope assemblies are bronchoscope assemblies.

In some instances, the endoscope assembly may include at least one ultrasound transducer between the angled side endoscope port and the distal endoscope port.

In some instances, the surgical instrument is configured for positioning in a working channel of the catheter to extend distally through a distal catheter port, the catheter extending from one of the angled side endoscope port or the distal endoscope port. In some instances, more than one surgical instrument may be used with the endoscope assemblies described herein.

In some embodiments, the present disclosure describes a catheter assembly configured for navigation within a luminal structure including at least a catheter and a surgical instrument. The catheter including a tube portion having a catheter sidewall defining a working channel therein, a distal catheter port positioned on a distal end of the tube portion and in communication with the working channel, and an angled side catheter port defined through a distal portion of the catheter sidewall and in communication with the working channel. The surgical instrument is configured for positioning in the working channel to extend distally through the distal catheter port or extend laterally through the angled side catheter port. In some instances, the catheter may be configured to be curved. In some instances, the surgical instrument may be pre-curved or include a fix curved, in particular an elliptical fixed curve. In some instances, the surgical instrument may be steerable or articulatable to form a curve.

The angled side catheter port is a compound opening. For example, the angled side catheter port includes two or more edges to define the port or opening. In some instances, the compound opening defines at least a first edge along the catheter sidewall and extending generally perpendicular to a longitudinal axis of the catheter channel and a second edge along the catheter sidewall and extending at an acute angle relative to the longitudinal axis of the catheter channel. In some instances, the first and second edges form a first angle therebetween ranging from about 25° to about 85° or from about 35° to about 75°.

In some instances, the catheter assembly may include at least one ultrasound transducer between the angled side catheter port and the distal catheter port.

In some instances, the catheter is configured for positioning in an endoscope channel of the endoscope to extend distally through a distal endoscope port, and the surgical instrument is configured for positioning in the catheter working channel to extend through one of the angled side catheter port or the distal catheter port. In some instances, more than one surgical instrument may be used with the catheter assemblies described herein.

In some embodiments, the endoscope assemblies and/or catheter assemblies described herein are configured to be used with electromagnetic navigation systems for navigating through a luminal network of a patient's lung. In some instances the system includes an endoscope assembly or bronchoscope assembly including at least one of an endoscope or bronchoscope including a distal endoscope port and an angled side endoscope port or a catheter including a distal catheter port and an angled side catheter port, and optionally or more surgical instruments. The systems may also include one or more of a computing device, a monitoring device, an electromagnetic board, and a tracking device.

### BRIEF DESCRIPTION OF THE DRAWINGS

Various aspects and features of the present disclosure are described herein below with reference to the drawings, wherein:

FIGS. 1A and 1B depict a side view of an endoscope assembly as described in at least one embodiment herein;

FIGS. 2A and 2B are schematic cross-sectional views of a portion of the endoscope assembly of FIGS. 1A and 1B, respectively, as described in at least one embodiment herein;

FIGS. 3A-3C depict schematic cross-sectional side views of various endoscopes as described in at least one embodiment herein;

FIGS. 4A-4D depict side views of various surgical instruments as described in at least one embodiment herein;

FIGS. 5A and 5B depict schematic top and side cross-sectional views, respectively, of an endoscope as described in at least one embodiment herein;

FIGS. 6A and 6B depict schematic top and side cross-sectional views, respectively, of an endoscope as described in at least one embodiment herein;

FIGS. 7A and 7B depict schematic top and side cross-sectional views, respectively, of an endoscope as described in at least one embodiment herein;

FIGS. 8A and 8B depict schematic top and side cross-sectional views, respectively, of an endoscope as described in at least one embodiment herein;

FIGS. 9A and 9B depict a side view of a catheter assembly as described in at least one embodiment herein;

FIGS. 10A and 10B are schematic cross-sectional side views of a portion of the catheter assembly of FIGS. 9A and 9B, respectively, as described in at least one embodiment herein;

FIGS. 11A-11C depict schematic cross-sectional side views of various catheters as described in at least one embodiment herein;

FIGS. 12A-12D depict side views of various surgical instruments as described in at least one embodiment herein;

FIGS. 13A and 13B depict schematic top and side cross-sectional views, respectively, of a catheter as described in at least one embodiment herein;

FIGS. 14A and 14B depict schematic top and side cross-sectional views, respectively, of a catheter as described in at least one embodiment herein;

FIGS. 15A and 15B depict schematic top and side cross-sectional views, respectively, of a catheter as described in at least one embodiment herein;

FIGS. 16A and 16B depict schematic top and side cross-sectional views, respectively, of a catheter as described in at least one embodiment herein;

FIG. 17 is a schematic perspective view of a navigation system for visualizing a lung of a patient as described in at least one embodiment herein;

FIG. 18A is an illustration of an endoscope assembly inserted into a lung as described in at least one embodiment herein; and

FIG. 18B is an enlarged detail view of the circled area of FIG. 18A.

### DETAILED DESCRIPTION

The present disclosure describes an endoscope assembly for navigation within a luminal structure including an endoscope and at least one catheter configured for positioning within at least a portion of the endoscope, wherein at least one of the endoscope or the catheter, individually includes at least two ports of exit, such as a distal end port and an angled side port defined therein. For example, in some embodiments, the endoscope assembly may include an endoscope having a distal endoscope port and an angled side endoscope port. In another example, in some embodiments, the endoscope assembly may include a catheter having a distal catheter port and an angled side catheter port. The endoscope assembly described herein may also further include at least one surgical instrument configured for positioning within a portion of the endoscope or the catheter.

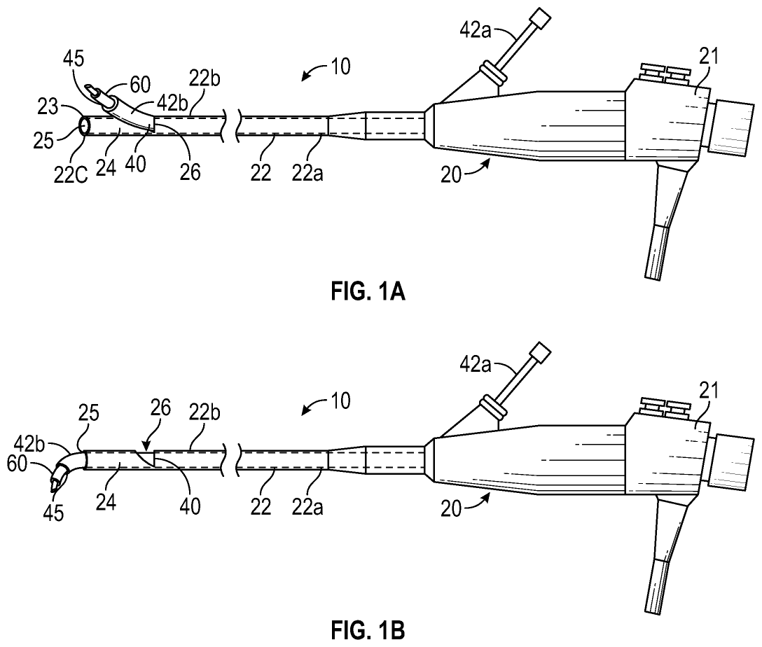

FIGS. 1A and 1B depict an endoscope assembly 10 including an endoscope 20, a catheter 40, and optionally a surgical instrument 60. The endoscope 20 includes an endoscope handle portion 21 and an endoscope shaft portion 22. The shaft portion 22 includes an endoscope sidewall 23 defining at least one endoscope channel 24 therein. The endoscope channel 24 extends from a proximal end portion 22a to a distal end portion 22b of the shaft portion 22. The endoscope channel 24 is configured to receive therein and/or therethrough at least a distal portion of at least one of, if not both, the catheter 40 or the surgical instrument 60.

A distal endoscope port 25 is positioned on the distal end 22c of the shaft portion 22 and in communication with the channel 24. An angled side endoscope port 26 is positioned through the endoscope sidewall 23 and proximal to the distal endoscope port 25 (or proximal to the distal end 22c of the shaft portion 22). The angled side endoscope port 26 is also in communication with the channel 24. The channel 24 is configured to receive or maintain at least one, if not both, of the catheter 40 or the surgical instrument 60.

In FIG. 1A, the catheter 40, and particularly a distal end portion 42b of the catheter 40, is shown extending through the angled side endoscope port 26 of the endoscope 20. In FIG. 1B, the catheter 40, and particularly the distal end portion 42b of the catheter 40, is shown extending through the distal endoscope port 25 of the endoscope 20. In FIGS. 1A and 1B, the surgical instrument 60 is shown extending from a distal catheter port 45 of the catheter 40, wherein the catheter 40 is configured to include a distal end portion 42b which is curved.

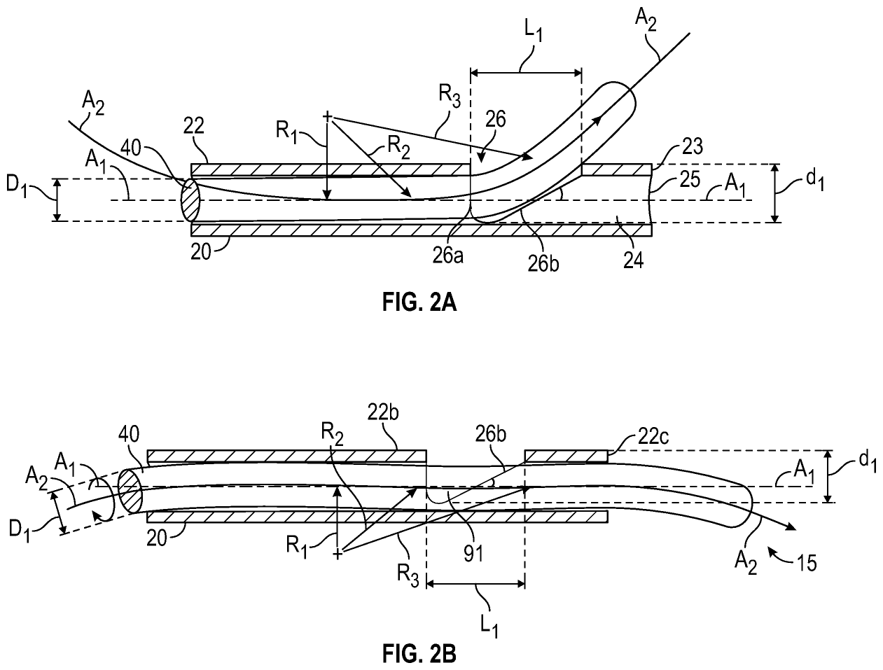

FIGS. 2A and 2B depict a schematic cross-sectional view of the distal end portion 22b of the shaft portion 22 of the endoscope 20, and the distal end portion 42b of the catheter 40 of FIGS. 1A and 1B, respectively. For clarity purposes the surgical instrument 60 is not shown in FIGS. 2A and 2B. FIGS. 2A and 2B further depict, in some embodiments, that the distal end portion 22b of the endoscope 20 may define a generally linear longitudinal axis A₁, i.e., does not include a fixed curvature, while at least the distal end portion 42b of the catheter 40 is configured to be curved, i.e., the catheter includes a fixed curvature or the catheter is steerable to form a curve. Although generally linear, the endoscope may be made from a flexible material which may flex into a non-linear configuration, however, a generally linear endoscope does not include a fixed curve, such as the catheter in some embodiments. In some embodiments, at least the distal end portion 42b of the catheter 40 is pre-curved, i.e., has a predefined radius or fixed curvature. In some embodiments, at least the distal end portion 42b of the catheter 40 includes two concentric steerable tubes that can be manipulated relative to each other to form a curve on the distal end portion 42b of the catheter 40, as may be known in the art. The surgical instrument 60 (not shown in FIG. 2A or 2B for clarity purposes) may define a generally linear longitudinal axis or a curved longitudinal axis.

In FIG. 2A, the curved distal end portion 42b of the catheter 40 may be positioned to extend from the endoscope channel 24 through the angled side endoscope port 26 into the tissue positioned adjacent a side of the distal end portion 22b of the endoscope 20. In FIG. 2B, the curved distal end portion 42b of the catheter 40 may be rotated (as indicated by the circular arrow) around the curved longitudinal axis A₂ to be positioned to extend from the endoscope channel 24 through the distal endoscope port 25 into the tissue positioned adjacent the distal end 22c of the endoscope 20.

As shown in FIGS. 2A and 2B, in some embodiments, at least a portion of the catheter 40, and particularly the distal end portion 42b, may include a curved longitudinal axis A₂ defining at least one radius R₁. In some embodiments, as best shown in FIG. 2A, the curved longitudinal axis A₂ may define a plurality, i.e., 2 or more, of radii R₁-R₃ wherein the radii increase in length distally, i.e., R₁<R₂<R₃ from a given point (*), to form an elliptical curve.

FIGS. 2A and 2B further illustrate the angled side endoscope port 26 defined through the endoscope sidewall 23. The angled side endoscope port 26 is a compound port, i.e., includes at least a first and second edge 26a, 26b to define the port 26. Unlike a simple port, i.e., including only a single edge commonly defined by a punch process, the compound port defines an opening in three dimensions of the sidewall 23 which improves the ability of a curved catheter to pass therethrough and/or to extend along the outer side of the endoscope (as shown in FIG. 2A). The first edge 26a extends generally perpendicular to the longitudinal axis A₁ of the endoscope 20 and the second edge 26b extends at an acute angle to the longitudinal axis of the endoscope 20. In some embodiments, the second edge 26b is concave (FIG. 2A). In some embodiments, the second edge 26b may be convex (FIG. 2B). The first and second edges 26a, 26b meet to define a first angle a₁. The first angle a₁: ranging from 20 to 80 degree; ranging from 25 to 75 degrees; ranging from 30 to 65 degrees; ranging from 35 to 55 degrees; or being 45 degrees.

The angled side endoscope port 26 defines an opening having length L₁ and depth d₁ in the sidewall 23. The length L₁ is greater than a diameter D1 of the catheter 40 and the depth d₁ represents at least half the outer circumference of the catheter 40. The use of the terms diameter and circumference are not intended to limit the catheter 40 and/or the port 26 to only a circular shape. Rather, the term diameter is intended to represent the widest or thickest part of the catheter generally transverse the longitudinal axis and the term circumference is intended to represent the outer perimeter of the catheter.

In some embodiments, the length L₁ of the angled side endoscope port 26 may be from 1.1 to 3 times greater than the diameter D₁ of the catheter 40. In some embodiments, the length L₁ of the angled side endoscope port 26 may be from 1.5 to 2.5 times greater than the diameter D₁ of the catheter 40.

In some embodiments, the depth d₁ of the angled side endoscope port 26 may represent from 50 to 75% of the outer circumference of the catheter 40. In some embodiments, the depth d₁ of the angled side endoscope port 26 may represent from 55 to 70% of the outer circumference of the catheter 40.

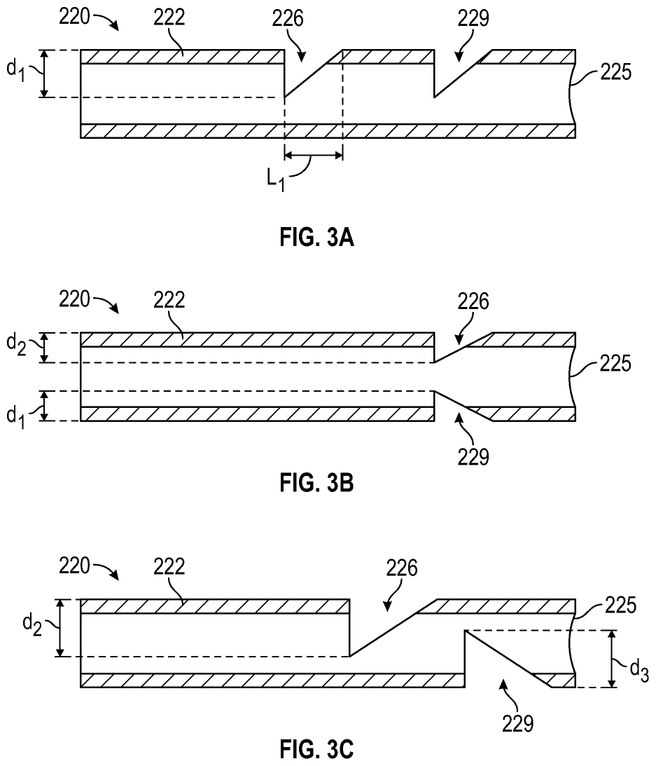

In some embodiments, as shown in FIGS. 3A-3C, an endoscope 220 described herein may include at least two angled side endoscope ports 226, 229 in various configurations and a distal end endoscope port 225. For example, FIG. 3A depicts an endoscope 220 including first and second angled side endoscope ports 226, 229 positioned on the same side of the shaft 222 with the first endoscope port 226 being proximal to the second endoscope port 229. In another example, FIG. 3B depicts an endoscope 220 including first and second endoscope ports 226, 229 positioned on opposite sides of the shaft 222 with the first endoscope port 226 being aligned longitudinally with the second endoscope port 229. When aligned longitudinally, as shown in FIG. 3B, it is worth noting that the depths d₂, d₃, as defined by the respective first edges 226a, 229a, of each port 226, 229 must represent less than half of the outer circumference of the endoscope 220, but collectively represents at least half of the outer circumference of the endoscope 220.

In still another example, as depicted in FIG. 3C, an endoscope 220 may include a first and second angled side endoscope ports 226, 229 positioned on opposite sides of the shaft 222 and staggered longitudinally, such that the first port 226 is proximal the second port 229. When staggered longitudinally, as shown in FIG. 3C, the depths d₂, d₃, as defined by the respective first edges 226a, 229a, of each port 226, 229 may represent at least half of the outer circumference of the endoscope.

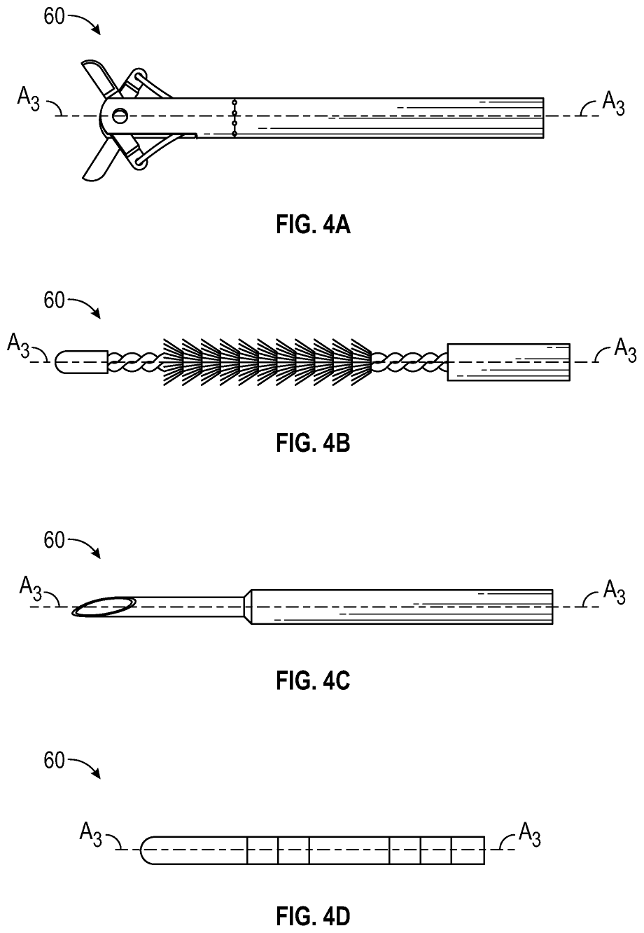

The endoscope assemblies described herein may also include a surgical instrument configured to be received within and/or pass through both the endoscope channel of the endoscopes described herein and the catheter channel of the catheters described herein. For example, the surgical instrument may be selected from the group consisting of a locating guide, an imaging device, a guidewire, a surgical balloon, a biopsy forceps, a cytology brush, an aspirating needle, an ablation device, and combinations thereof. Some examples of suitable surgical instruments are depicted in FIGS. 4A-4D, including for example, a biopsy forceps (FIG. 4A), a cytology brush (FIG. 4B), an aspirating needle (FIG. 4C), and an ablation catheter (FIG. 4D) may be inserted into the endoscope assemblies or endoscopes described herein to obtain a tissue sample from the target tissue or to treat target tissue as appropriate. As further depicted, in some embodiments, at least the distal end portion of the surgical instruments generally define a linear longitudinal axis A3 and does not include a curved longitudinal axis on the distal end thereof.

Although FIGS. 1A-2B depict the endoscope assembly 10 including a catheter 40 extending from the endoscope 20, it is further envisioned that in some embodiments the endoscope assembly 10 may only further include a surgical instrument 60 as described herein. The surgical instrument extending directly from the angled side endoscope port 26 or distal endoscope port 25 without the catheter 40 positioned between the endoscope 20 and the surgical instrument 60.

In such embodiments, an endoscope assembly for navigation within a luminal structure is described as including an endoscope with a shaft having an endoscope sidewall defining an endoscope channel therein, a distal endoscope port positioned on a distal end of the shaft and in communication with a distal end of the endoscope channel, and an angled side endoscope port defined through a distal portion of the endoscope sidewall and in communication with the endoscope channel, and a surgical instrument configured for positioning in the endoscope channel to extend distally through the distal endoscope port or extend laterally through the angled side endoscope port. The surgical instrument may define either a linear longitudinal axis or a curved axis.

In addition to the multiple exit ports, the endoscopes or endoscope assemblies described herein may further include one or more ultrasound (US) transducers on a distal end portion thereof. The one or more US transducers are configured to transmit ultrasound waves and/or receive reflected ultrasound waves. Generally, the ultrasound waves penetrate the tissue surrounding the distal end portion of the endoscope based on the frequency of the ultrasound waves. For example, 1 megahertz (MHz) ultrasound waves penetrate to a depth of 2 cm to 5 cm and 3 MHz ultrasound waves penetrate to a depth of 1.5 cm.

Generally, the US waves are reflected at a boundary where density changes or at the interface between tissues. During the navigation process, such as navigating the luminal network of the lung, the US waves are reflected from the inside wall of a bronchial tree, from the outside wall of the bronchial tree, and from a diseased portion or cancerous portion located at the outside wall of the bronchial tree and provide finite details of the lung structure and the tissue patency that could not otherwise be revealed using non-invasive imaging means. The reflected US waves have information such as amplitude and a delayed time between transmission of the US waves and reception of the reflected US waves. Since the US waves travels differently and attenuates amplitudes differently in accordance with the density of tissue, the amplitude and the delayed time may be used to identify a type of tissue, a density of the tissue, and/or a size of the tissue. Since the density of abnormal tissues (e.g., diseased or cancerous cells) are different from the normal lung tissue, the reflected US waves may be used to identify the diseased or cancerous cells from normal cells and the size and/or thickness of the diseased or cancerous cells.

In addition, after the navigation process is complete, the US transducer can be used to identify at least one of the distal end portion of the catheter or the distal end portion of surgical instrument, extending through one of the ports of the endoscope or endoscope assemblies described herein. In some embodiments, the US transducer may be positioned distal to the angled side endoscope port. Any suitable US transducer may be used. Some non-limiting examples include a radial transducer, a linear transducer, a piezoelectric transducer, and the like.

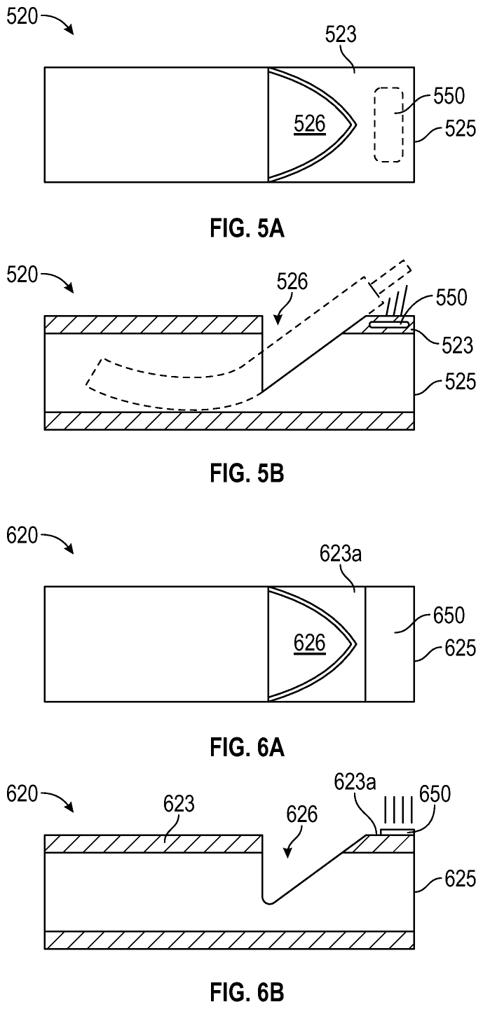

FIGS. 5A-8B include top views and cross-sectional views of various embodiments including the endoscopes including two or more exit ports as described herein. FIGS. 5A and 5B depict an endoscope 520 including at least an angled side endoscope port 526, a distal end endoscope port 525, and a US transducer 550 located distally to the angled side endoscope port 526 and defined within a portion of the sidewall 523. FIG. 5A illustrates a benefit of the various endoscopes of FIGS. 5A-8B which includes the ability to treat and/or biopsy the tissue under direct ultrasound. As specifically shown in FIG. 5A, the US transducer 550 will be positioned directly beneath a catheter and/or surgical instrument (shown in phantom) when extending from the angled side endoscope port 525 into the surrounding tissue 515.

FIGS. 6A and 6B depict an endoscope 620 including at least an angled side endoscope port 626, a distal end endoscope port 625, and a US transducer 650 distal the angled side endoscope port 626 and positioned on an outer surface 623a of the endoscope sidewall 623.

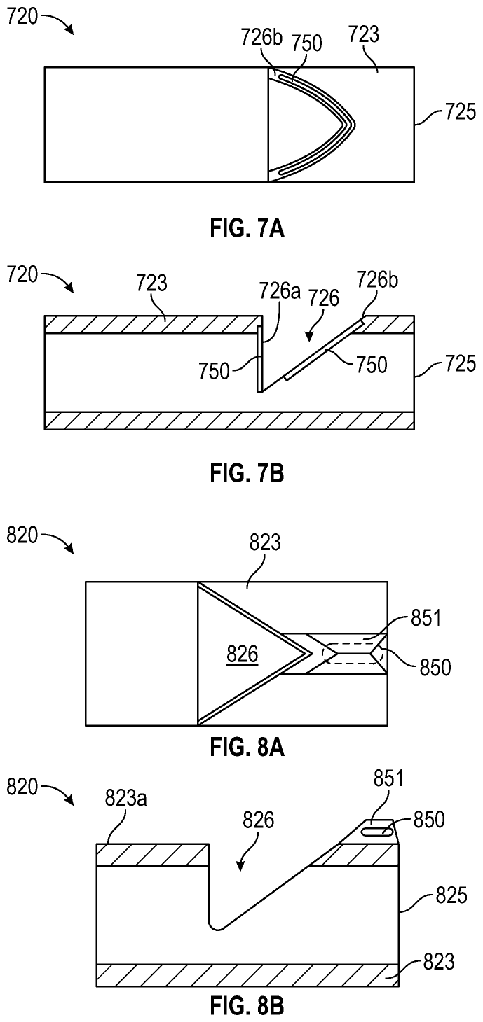

FIGS. 7A and 7B depict an endoscope 720 including at least distal an angled side endoscope port 726, a distal end endoscope port 725, and one or more US transducers 750 defined within a portion of the sidewall 723 along at least one of the first or second edges 726a, 726b of the angled side endoscope port 726.

FIGS. 8A and 8B depict an endoscope 820 including at least distal an angled side endoscope port 826, a distal end endoscope port 825, and one or more US transducers 850 positioned on an exterior surface 823a of the endoscope sidewall 823. The US transducer 850 either defines or is encased in a ramp 851 configured to support and/or guide a catheter and/or surgical instrument after exiting the endoscope channel 824 through the angled side endoscope port 826. The ramp or support 851 is generally centered on the second edge 826b of the angled side port 826 thereby essentially extending the second edge 826b beyond the sidewall 823. In each of these embodiments, the US transducer is configured to be positioned directly beneath a catheter and/or a surgical instrument when extending out of the angled side endoscope port making it possible treat and/or biopsy the surrounding tissue under direct ultrasound imaging.

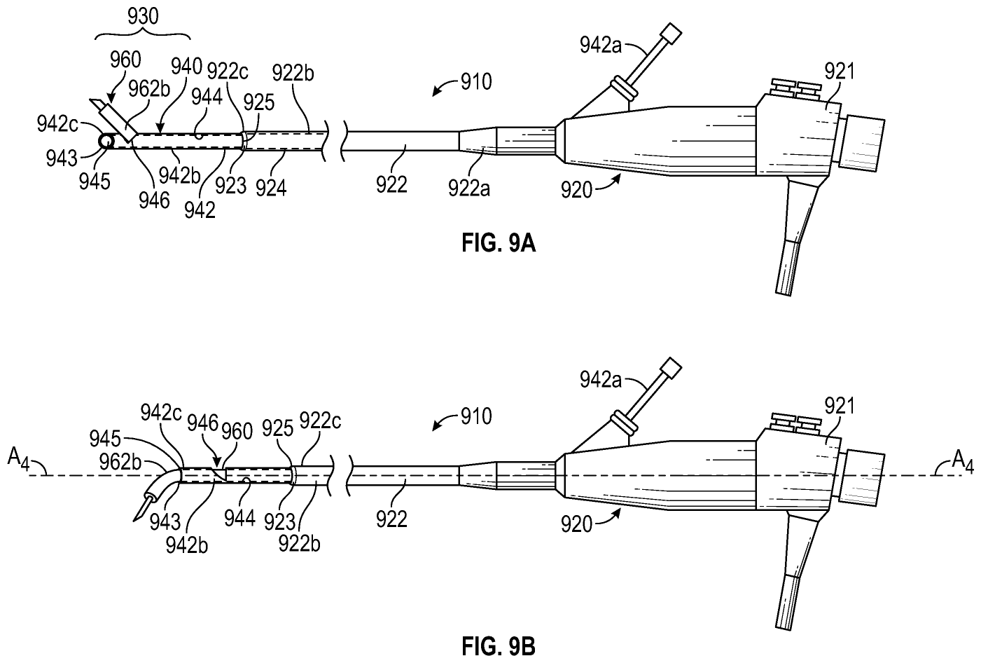

FIGS. 9A and 9B depict another endoscope assembly 910 including an endoscope 920, a catheter 940 and optionally a surgical instrument 960. In some embodiments, the endoscope assembly 910 includes an endoscope 920 including a single exit port 925 on a distal end portion 922b thereof and a catheter assembly 930, the catheter assembly 930 including a catheter 940 including two or more exit ports 945, 946 on a distal end portion thereof 942b and a surgical instrument 960.

The endoscope 920 of FIGS. 9A and 9B includes an endoscope handle portion 921 and an endoscope shaft portion 922. The shaft portion 922 includes an endoscope sidewall 923 defining an endoscope channel 924 therein. The channel 924 extends from a proximal end portion 922a to a distal end portion 922b of the shaft portion 922. The endoscope channel 924 is configured to receive therein and/or therethrough at least a distal portion of at least one of, if not both, the catheter 940 or the surgical instrument 960.

A distal endoscope port 925 is positioned on the distal end 922c of the shaft portion 922 and in communication with the channel 924. The endoscope 920 as shown, in some embodiments, does not include an angled side endoscope port.

The catheter assembly 930 of FIGS. 9A and 9B includes a catheter 940 with a tube 942 including a catheter sidewall 943 defining at least one catheter channel 944 therein. The catheter channel 944 is an extended working channel extending from a proximal end portion 942a to a distal end portion 942b, and particularly a most distal end 942c, of the tube 942. The catheter channel 944 is configured to receive therein and/or therethrough at least a distal end portion 962b of the surgical instrument 960.

A distal catheter port 945 is positioned on the distal end 942c of the tube 942 and in communication with the catheter channel 944. An angled side catheter port 946 is positioned through catheter sidewall 943 and positioned proximally to the distal catheter port 945 (or proximal to the distal end 942c of the tube 942). The angled side catheter port 946 is also in communication with the catheter channel 944. The catheter channel 944 is configured to receive or maintain at least one surgical instrument 960.

In FIG. 9A, the surgical instrument 960, and particularly a distal end portion 962b of the surgical instrument 960, is shown extending through the angled side catheter port 946 of the catheter 940. In FIG. 9B, the surgical instrument 960, and particularly a distal end portion 962b of the surgical instrument 960, is shown extending through the distal catheter port 945 of the catheter 940. In FIGS. 9A and 9B, the catheter 940 is shown extending from a distal endoscope port 925 of the endoscope 920, wherein the endoscope 920 defines a generally linear longitudinal axis A₄. In some embodiments, the tube 942 of the catheter 940 may also define a generally linear longitudinal axis A₅, while the surgical instrument 960 includes a distal end portion 962b which is curved and defines a curved longitudinal axis A₆. Although generally linear, the endoscope or catheter may be made from a flexible material which may flex into a non-linear configuration, however, a generally linear endoscope or catheter does not include a fixed curve, such as the surgical instrument in some embodiments.

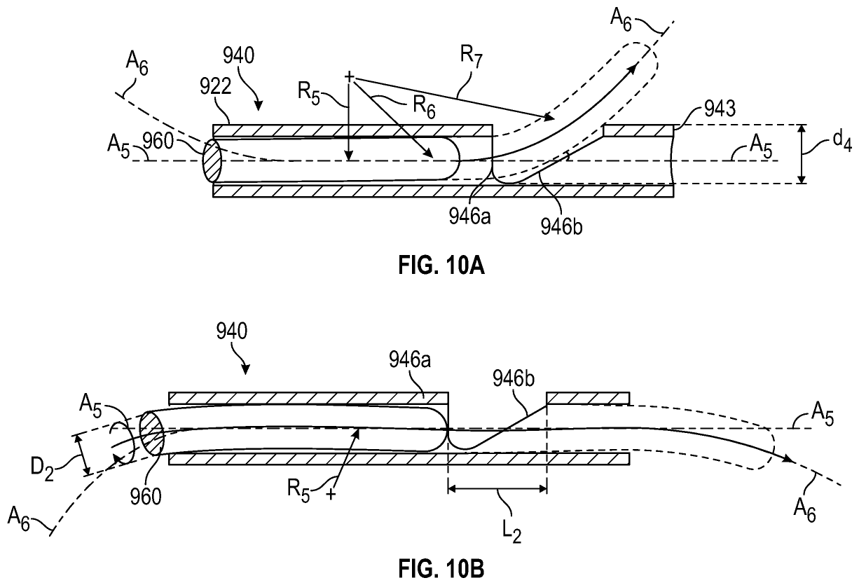

FIGS. 10A and 10B depict a schematic cross-sectional view of the distal end portion 942b of the catheter 940 and the distal end portion 962b surgical instrument 960 of FIGS. 9A and 9B, respectively. For clarity purposes the endoscope 920 is not shown in FIGS. 10A and 10B. FIGS. 10A and 10B further depict, in some embodiments, that the distal end portion 942b of the catheter 940 may define a generally linear longitudinal axis A₅, i.e., does not include a fixed curvature, while at least the distal end portion 962b of the surgical instrument 960 is configured to be curved, i.e., the instrument includes a fixed curvature or the catheter is steerable to form a curve. In some embodiments, the surgical instrument 960 is pre-curved, i.e., has a natural radius or fixed curvature. In some embodiments, the surgical instrument 960 is two concentric steerable tubes that can be manipulated relative to each other to form a curve as known in the art.

In FIG. 10A, the curved distal end portion 962b of the surgical instrument 960 may be positioned to extend from the catheter channel 944 through the angled side catheter port 946 into the tissue positioned adjacent a side of the distal end portion 942b of the catheter 940. In FIG. 10B, the curved distal end portion 962b of the surgical instrument 960 may be rotated around the curved longitudinal axis A₆ (as indicated by the circular arrow) to be positioned to extend from the catheter channel 944 through the distal catheter port 945 into the tissue positioned adjacent the distal end 942c of the catheter 940.

As shown in FIGS. 10A and 10B, in some embodiments, at least a portion of the surgical instrument 960, and particularly the distal end portion 962b, may include a curved longitudinal axis A₆ defining at least one radius R₅. In some embodiments, as best shown in FIG. 10A, the curved longitudinal axis A₆ may define a plurality, i.e., 2 or more, of radii R₅-R₇ wherein the radii increase in length distally, i.e., R₅<R₆<R₇ from a given point (*), to form an elliptical curve.

FIGS. 10A and 10B further illustrate the angled side catheter port 946 defined through the catheter sidewall 943. The angled side catheter port 946 is a compound port, i.e., includes at least a first and second edge 946a, 946b to define the port 946. Unlike a simple port, i.e., including only a single edge commonly defined by a punch process, the compound port defines an opening in three dimensions of the sidewall 943 which improves the ability of a curved surgical instrument to pass therethrough and/or to extend along the outer side of the endoscope (as shown in FIG. 10A). The first edge 946a extends generally perpendicular to the longitudinal axis A₅ of the catheter 940 and the second edge 946b extends at an acute angle to the longitudinal axis A₅ of the catheter 940. In some embodiments, the second edge 946b is concave (FIG. 10A). In some embodiments, the second edge 946b may be convex (FIG. 10B). The first and second edges 946a, 946b meet to define an angle a₂. The angle a₂ being: 20 to 80 degree; 25 to 75 degrees; 30 to 65 degrees; 35 to 55 degrees; or 45 degrees.

The angled side catheter port 946 defines an opening having length L₂ and depth d₂ in the sidewall 943. The length L₂ is greater than a diameter D₂ of the surgical instrument 960 and the depth d₄ represents at least half the outer circumference of the surgical instrument 960. The use of the terms diameter and circumference are not intended to limit the surgical instrument 960 or the port 946 to only a circular shape. Rather, the term diameter is intended to represent the widest or thickest cross-sectional part of the surgical instrument generally transverse the longitudinal axis A₆ and the term circumference is intended to represent the outer perimeter of the surgical instrument.

In some embodiments, the length L₂ of the angled side catheter port 926 may be from 1.1 to 3 times greater than the diameter D₂ of the surgical instrument 960. In some embodiments, the length L₂ of the angled side catheter port 926 may be from 1.5 to 2.5 times greater than the diameter D₂ of the surgical instrument 960.

In some embodiments, the depth d₄ of the angled side catheter port 926 may represent from 50 to 75% of the outer circumference of the surgical instrument 960. In some embodiments, the depth d₄ of the angled side catheter port 926 may represent from 55 to 70% of the outer circumference of the surgical instrument 960.

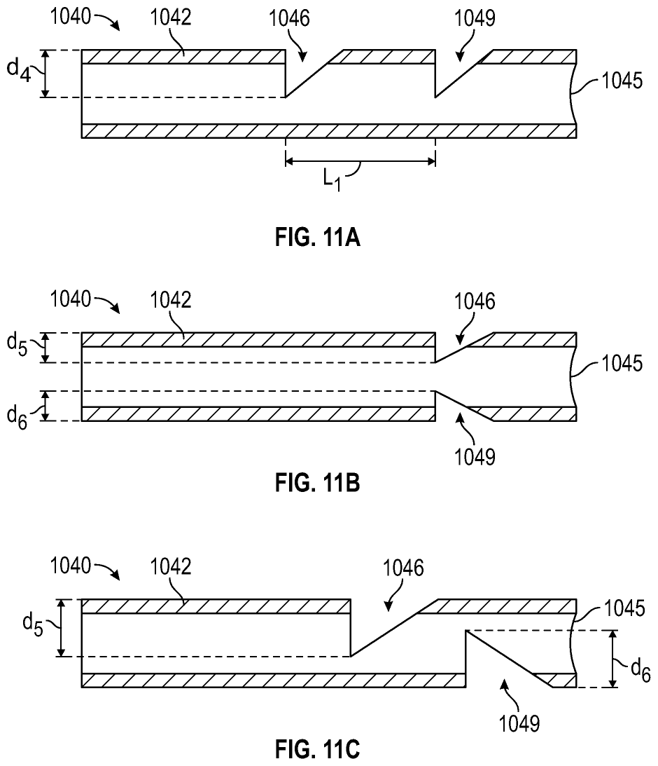

In some embodiments, as shown in FIGS. 11A-11C, a catheter 1040 as described herein may include at least two angled side catheter ports 1046, 1049 and a distal end endoscope port 1025. For example, FIG. 11A depicts a catheter 1040 including first and second angled side catheter ports 1046, 1049 positioned on the same side of the tube 1042 with the first angled side catheter port 1046 being proximal to the second angled side catheter port 1049. In another example, FIG. 11B depicts a catheter 1040 including first and second angled side catheter port 1046, 1049 positioned on opposite sides of the tube 1042 with the first catheter port 1046 being aligned longitudinally with the second catheter port 1049. When aligned longitudinally, as shown in FIG. 11B, the depths d₅, d₆ of the two first edges 1046a, 1049a of the angled side ports 1046, 1049, respectively, may individually represent less than half the outer circumference of the catheter and/or may collectively represent at least half the outer circumference of the catheter.

In still another example, as depicted in FIG. 11C, a catheter 1040 including first and second angled side catheter ports 1046, 1049 positioned on opposite sides of the tube 1042 with the first catheter port 1046 being staggered longitudinally, such that the first port 1046 is proximal to the second port 1049 with the second catheter port 1049. When staggered longitudinally, as shown in FIG. 11C, the depths d₅, d₆ of the two first edges 1046a, 1049a of the angled side ports 1046, 1049, respectively, may individually represent at least half the outer circumference of the catheter.

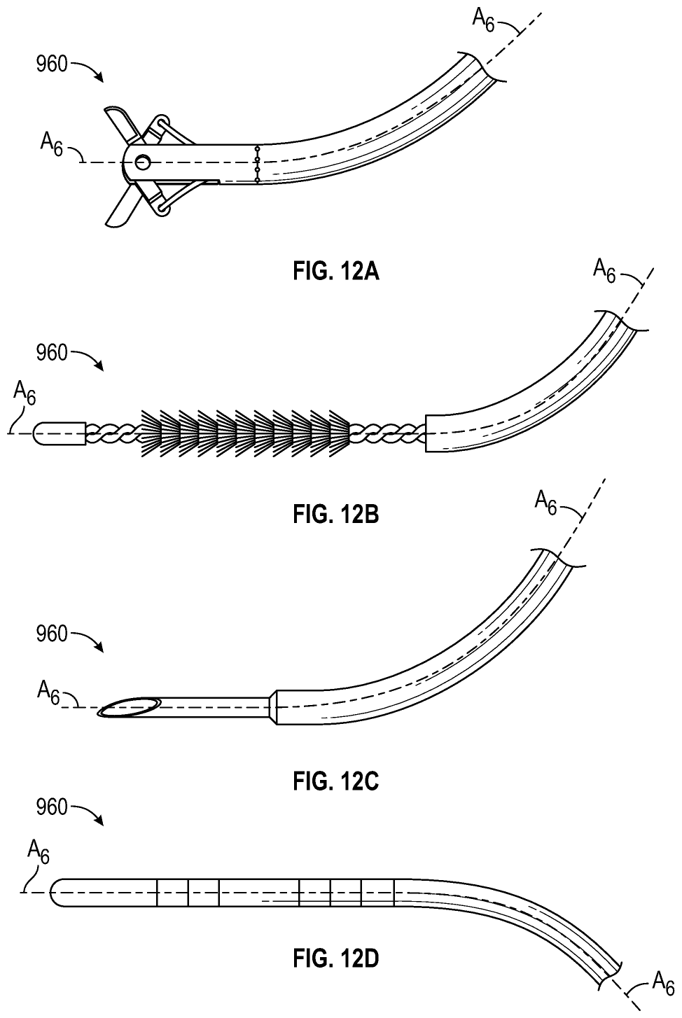

The catheter assemblies described herein may also include a surgical instrument configured to be received within and/or pass through the catheter channel of the catheters described herein. For example, the surgical instrument may be selected from the group consisting of a locating guide, an imaging device, a guidewire, a surgical balloon, a biopsy forceps, a cytology brush, an aspirating needle, an ablation device, and combinations thereof. Some examples are depicted in FIGS. 12A-12D, including for example, a biopsy forceps (FIG. 12A), a cytology brush (FIG. 12B), an aspirating needle (FIG. 12C), and an ablation catheter (FIG. 12D) may be inserted into the catheter assemblies described herein to obtain a tissue sample from the target tissue or to treat target tissue as appropriate. As further depicted, in some embodiments, at least the distal end portion of the surgical instruments generally define a curved longitudinal axis A₆ and does not include a linear longitudinal axis on the distal end thereof.

In addition to the multiple exit ports, the catheters or catheter assemblies described herein may further include one or more ultrasound (US) transducers on a distal end portion thereof. The one or more US transducers are configured to transmit ultrasound waves and/or receive reflected ultrasound waves. Generally, the ultrasound waves penetrate the tissue surrounding the distal end portion of the catheter based on the frequency of the ultrasound waves. For example, 1 megahertz (MHz) ultrasound waves penetrate to a depth of 2 cm to 5 cm and 3 MHz ultrasound waves penetrate to a depth of 1.5 cm.

Generally, the US waves are reflected at a boundary where density changes or at the interface between tissues. During the navigation process, such as navigating the luminal network of the lung, the US waves are reflected from the inside wall of a bronchial tree, from the outside wall of the bronchial tree, and from a diseased portion or cancerous portion located at the outside wall of the bronchial tree and provide finite details of the lung structure and the tissue patency that could not otherwise be revealed using non-invasive imaging means. The reflected US waves have information such as amplitude and a delayed time between transmission of the US waves and reception of the reflected US waves. Since the US waves travels differently and attenuates amplitudes differently in accordance with the density of tissue, the amplitude and the delayed time may be used to identify a type of tissue, a density of the tissue, and/or a size of the tissue. Since the density of abnormal tissues (e.g., diseased or cancerous cells) are different from the normal lung tissue, the reflected US waves may be used to identify the diseased or cancerous cells from normal cells and the size and/or thickness of the diseased or cancerous cells.

In addition, after the navigation process is complete, the US transducer can be used to identify at least the distal end portion of surgical instrument, extending through one of the ports of the catheter or catheter assemblies described herein. In some embodiments, the US transducer may be positioned distal to the angled side catheter port. Any suitable US transducer may be used. Some non-limiting examples include a radial transducer, a linear transducer, a piezoelectric transducer, and the like.

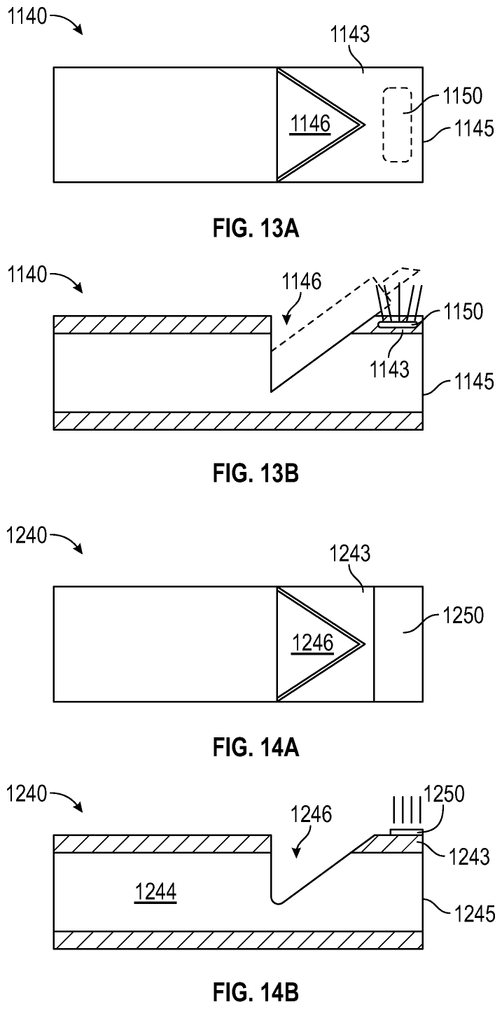

FIGS. 13A-16B are cross-sectional views of various embodiments including the catheters including two or more exit ports as described herein. FIGS. 13A and 13B depict a catheter 1140 including at least an angled side catheter port 1146, a distal end catheter port 1145, and a US transducer 1150 located distally to the angled side catheter port 1146 and defined within a portion of the sidewall 1143. FIG. 13A illustrates a benefit of the various catheters of FIGS. 13A-16B which includes the ability to treat and/or biopsy the tissue under direct ultrasound analysis. As schematically shown in FIG. 13B, the US transducer 1150 is positioned directly beneath a second inner catheter and/or surgical instrument (shown in phantom) when extending from the angled side catheter port 1125 into the surrounding tissue.

FIGS. 14A and 14B depict a catheter 1240 including at least distal an angled side catheter port 1246, a distal end catheter port 1245, and a US transducer 1250 distal the angled side catheter port 1246 and positioned on an outer surface of the catheter sidewall 1243.

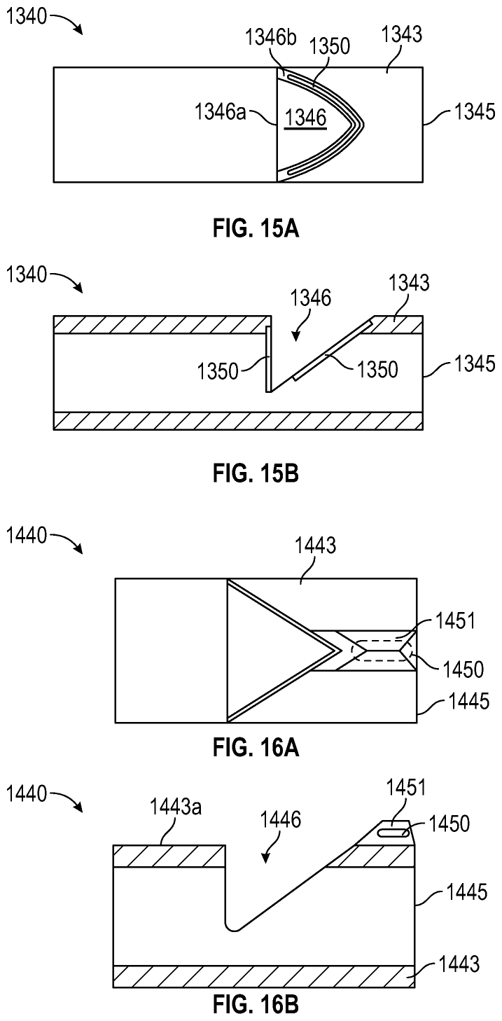

FIGS. 15A and 15B depict a catheter 1340 including at least an angled side catheter port 1346, a distal end endoscope port 1345, and one or more US transducers 1350 positioned on and/or within at least one of the first or second edges 1346a, 1346b of the angled side catheter port 1346.

FIGS. 16A and 16B depict a catheter 1440 including at least distal an angled side catheter port 1446, a distal end catheter port 1445, and one or more US transducers 1450 positioned on an exterior surface 1443a of the catheter sidewall 1443. The US transducer 1450 either defines or is encased in a ramp 1451 configured to support and/or guide a catheter and/or surgical instrument after exiting the catheter channel 1444 through the angled side catheter port 1446. The ramp or support 1451 is generally centered on the second edge 1446b of the angled side port 1446 thereby essentially extending the second edge 1446b beyond the sidewall 1443. In each of these embodiments, the US transducer is configured to be positioned directly beneath a catheter and/or a surgical instrument when extending out of the angled side catheter port making it possible treat and/or biopsy the surrounding tissue under direct ultrasound imaging.

The endoscopes described herein may be formed using any suitable method and/or any suitable biocompatible material known to those of ordinary skill. Some non-limiting examples of methods of forming the endoscope, and particularly at least the shaft portion of the endoscope, include extrusion, molding, casting, pressing, and the like.

In some embodiments, the endoscopes described herein may be manufactured by: forming a shaft portion of an endoscope, the shaft portion including a sidewall defining an endoscope channel therethrough and including at least a distal end port in communication with the endoscope channel; performing a first cut into a distal end portion of the shaft portion to form first edge of an angled side port, the first edge extending generally perpendicular to a longitudinal axis of the shaft portion and having a first depth; performing a second cut into the distal end portion of the shaft portion to form a second edge of the angled side port, the second edge meeting the first edge to form an acute angle therebetween to form the angled side endoscope port. In some embodiments, the second cut begins distal to the first cut and extends proximally towards the depth of the first cut.

The first and second cutting steps can be performed using any suitable cutting means including, but not limited to, using a laser, ultrasonics, a straight or curved blade, and combinations thereof. In some embodiments, the cutting means is moved relative to the shaft portion of the endoscope to form at least one of the first or second edges. In some embodiments, the shaft portion of the endoscope is moved relative to the cutting means to form at least one of the first or second edges.

The catheters described herein may be formed using any suitable method and/or any suitable biocompatible material known to those of ordinary skill. Some non-limiting examples of methods of forming the catheter, and particularly at least the tube portion of the catheter, include extrusion, molding, casting, pressing, and the like.

In some embodiments, the catheters described herein may be manufactured by: forming a tube portion of an catheter, the tube portion including a sidewall defining a channel therethrough and including at least a distal end port in communication with the channel; performing a first cut into a distal end portion of the tube portion to form first edge of an angled side port, the first edge extending generally perpendicular to a longitudinal axis of the tube portion and having a first depth; performing a second cut into the distal end portion of the tube portion to form a second edge of the angled side port, the second edge meeting the first edge to form an acute angle therebetween to form the angled side catheter port. In some embodiments, the second cut begins distal to the first cut and extends proximally towards the depth of the first cut.

The first and second cutting steps can be performed using any suitable cutting means including, but not limited to, using a laser, ultrasonics, a straight or curved blade, and combinations thereof. In some embodiments, the cutting means is moved relative to the tube portion of the catheter to form at least one of the first or second edges. In some embodiments, the tube portion of the catheter is moved relative to the cutting means to form at least one of the first or second edges.

The endoscope assemblies and/or catheter assemblies as described herein are configured to be used with systems for visualizing a luminal network of a patient, and/or particularly a lung of a patient. The systems, endoscope assemblies, and/or catheter assemblies as described herein may use ultrasound (US) imaging technologies which provide a sufficient resolution to identify and locate a target for diagnostic, navigation, and treatment purposes. US imaging, particularly in conjunction with non-invasive imaging, can provide a greater resolution and enable luminal network mapping and target identification. Further, additional clarity is provided with respect to tissue adjacent the endoscope, catheter, or identified targets which can result in different treatment options being considered to avoid adversely affecting the adjacent tissue.

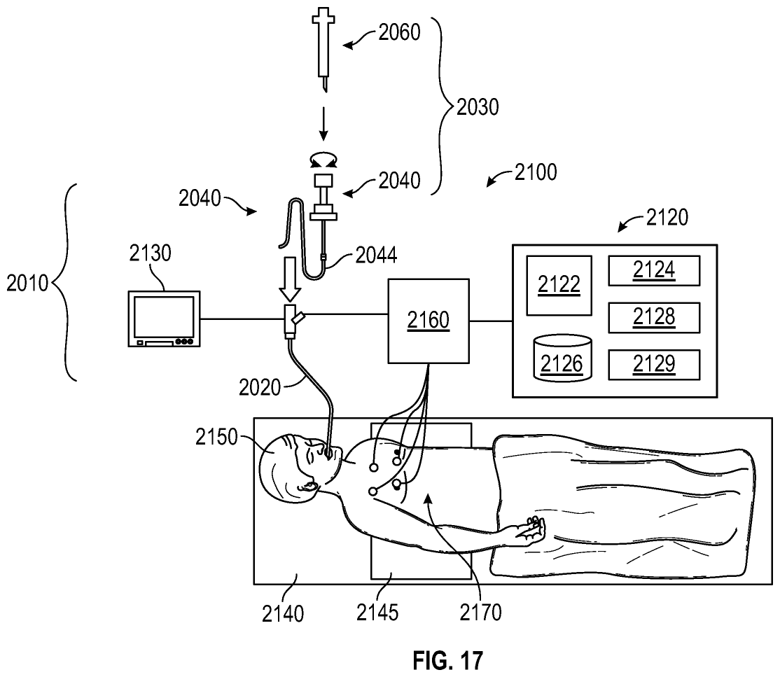

FIG. 17 illustrates an electromagnetic navigation (EMN) system 2100, which is configured to augment CT, MRI, or fluoroscopic images, with US image data assisting in navigation through a luminal network of a patient's lung to a target. One such EMN system may be the ELECTROMAGNETIC NAVIGATION BRONCHOSCOPY® system currently sold by Covidien LP. The system 2100 includes an endoscope assembly 2010 including an endoscope 2020, a catheter 2040, a surgical instrument 2060, a computing device 2120, a monitoring device 2130, an EM board 2140, a tracking device 2160, and reference sensors 2170. The endoscope 2020 is specifically a bronchoscope which is operatively coupled to the computing device 2120 and the monitoring device 2130 via wired connection (as shown in FIG. 17) or wireless connection (not shown).

The bronchoscope 2020 is inserted into the mouth of the patient 2150 and captures images of the luminal network of the lung. In the EMN system 2100, inserted into the bronchoscope 2020 is a catheter 2040 for achieving access to the periphery of the luminal network of the patient 2150. The catheter 2040 may include an extended working channel (EWC) 2044 into which surgical instrument 2060 may be inserted. A first surgical instrument, such as a locatable guide including an EM sensor at the distal tip thereof, may be inserted into the EWC 2044 to help navigate through the luminal network of the lung as described in greater detail below. Upon arrival of a desired location in the lung, the locatable guide may be removed from the EWC and replaced with a second surgical instrument configured to treat or biopsy a portion of the lung. As described herein, the endoscope or bronchoscope 2020 and/or the catheter 2040 may individually include two or more exit ports, i.e., an angled side port and a distal end port. As further described herein, the catheter 2040 and/or the surgical instrument 2060 may individually be curved or be configured to include a fixed curve for passage through the angled side port of the endoscope or catheter, respectively.

The computing device 2120, such as, a laptop, desktop, tablet, or other similar computing device, includes a display 2122, one or more processors 2124, memory 2126, a network card 2128, and an input device 2129. The system 2100 may also include multiple computing devices, wherein the multiple computing devices 2120 are employed for planning, treatment, visualization, or helping clinicians in a manner suitable for medical operations. The display 2122 may be touch-sensitive and/or voice-activated, enabling the display 2122 to serve as both an input and output device. The display 2122 may display a two dimensional (2D) images or three dimensional (3D) model of a luminal network, such as found in the lung, to locate and identify a portion of the network that displays symptoms of disease, such as lung disease. The generation of such images and models is described in greater detail below. The display 2122 may further display options to select, add, and remove a target to be treated and settable items for the visualization of the network or lung. In an aspect, the display 2122 may also display the location of the catheter 2040 or catheter assembly 2030 in the luminal network of the lung based on the 2D images or 3D model of the lung. For ease of description not intended to be limiting on the scope of this disclosure, a 3D model is described in detail below but one of skill in the art will recognize that similar features and tasks can be accomplished with 2D models and images.

The one or more processors 2124 execute computer-executable instructions. The processors 2124 may perform image-processing functions so that the 3D model of the lung can be displayed on the display 2122. In embodiments, the computing device 2120 may further include a separate graphic accelerator (not shown) that performs only the image-processing functions so that the one or more processors 2124 may be available for other programs.

The memory 2126 stores data and programs. For example, data may be image data for the 3D model or any other related data such as patients' medical records, prescriptions and/or history of the patient's diseases. One type of programs stored in the memory 2126 is a 3D model and pathway planning software module (planning software). An example of the 3D model generation and pathway planning software may be the ILOGIC® planning suite currently sold by Covidien LP. When image data of a patient, which is typically in digital imaging and communications in medicine (DICOM) format, from for example a CT image data set (or image data set by other imaging modality) is imported into the planning software, a 3D model of the bronchial tree is generated. In an aspect, imaging may be done by CT imaging, magnetic resonance imaging (MRI), functional MRI, X-ray, and/or any other imaging modalities. To generate the 3D model, the planning software employs segmentation, surface rendering, and/or volume rendering. The planning software then allows for the 3D model to be sliced or manipulated into a number of different views including axial, coronal, and sagittal views that are commonly used to review the original image data. These different views allow the user to review all of the image data and identify potential targets in the images.

Once a target is identified, the software enters into a pathway planning module. The pathway planning module develops a pathway plan to achieve access to the targets and the pathway plan pin-points the location and identifies the coordinates of the target such that they can be arrived at using the EMN system 2100 in combination with any of the endoscope or catheter assemblies described herein, and particularly the catheter 2040 or catheter assembly 2030 together with the EWC 2044 and a surgical instrument 2060 such as the locatable guide 2060. The pathway planning module guides a clinician through a series of steps to develop a pathway plan for export and later use in during navigation to the target in the patient 2150. The term, clinician, may include doctor, surgeon, nurse, medical assistant, or any user of the pathway planning module involved in planning, performing, monitoring and/or supervising a medical procedure.

The memory 2126 may store navigation and procedure software which interfaces with the EMN system 2100 to provide guidance to the clinician and provide a representation of the planned pathway on the 3D model and 2D images derived from the 3D model. An example of such navigation software may be the ILOGIC® navigation and procedure suite sold by Covidien LP. In practice, the location of the patient 2150 in the EM field generated by the EM field generating device 2145 must be registered to the 3D model and the 2D images derived from the model. Such registration may be manual or automatic.

As further shown in FIG. 17, the EM board 2140 is configured to provide a flat surface for the patient to lie down and includes an EM field generating device 2145. When the patient 2150 lies down on the EM board 2140, the EM field generating device 2145 generates an EM field sufficient to surround a portion of the patient 2150. An EM sensor on a distal tip of the LG 2060 may be used to determine the location of the LG 2060 in the EM field generated by the EM field generating device 2145.

In some embodiments, the EM board 2140 may be configured to be operatively coupled with the reference sensors 2170 which are located on the chest of the patient 2170. The reference sensors 2170 move up and down following the chest while the patient 2150 is inhaling and move down following the chest while the patient 2150 is exhaling. The movement of the reference sensors 2170 in the EM field is captured by the reference sensors 2170 and transmitted to the tracking device 2160 so that the breathing pattern of the patient 2150 may be recognized. The tracking device 2160 also receives outputs of the EM sensor on the LG 2060, combines both outputs, and compensates the breathing pattern for the location of the LG 2060. In this way, the location identified may be compensated for so that the compensated location of the LG 2060 is synchronized with the 3D model of the lung. Once the patient 2150 is registered to the 3D model, the position of the EWC 2044 (of the endoscope or catheter assemblies described herein) and particularly the LG 2060 can be tracked within the EM field generated by the EM field generator 2145, and the position of the LG 2060 can be depicted in the 3D model or 2D images of the navigation and procedure software.

When the endoscope 2020 or catheter 2040, and the LG 2060, reaches a target tissue by following the pathway plan, the LG 2060 including the EM sensor confirms its location at the target and a clinician may confirm the location at the target. The LG 2060 may be removed from the catheter 2040 and/or endoscope 2020 and a second surgical instrument 2060 such as biopsy tool may be inserted into the EWC 2044 to the target to retrieve sample of the target for confirmation of the disease. Further, or alternatively, treatment tools such as an ablation catheter may be inserted through the EWC 2044 and into the target. Any of the surgical instruments used to navigate, biopsy, or treat the target may extend through either of the two exits ports of the endoscopes or catheters described herein. Any US transducers included with the distal end portion of the endoscope or catheter as described herein may then be used to transmit and receive US waves and the computing device 120 determines whether the treatment tool is properly situated relative to the distal end portion of the endoscope or catheter, as well as to the target. By being properly aligned, the biopsy or treatment tool may perform with higher efficiency.

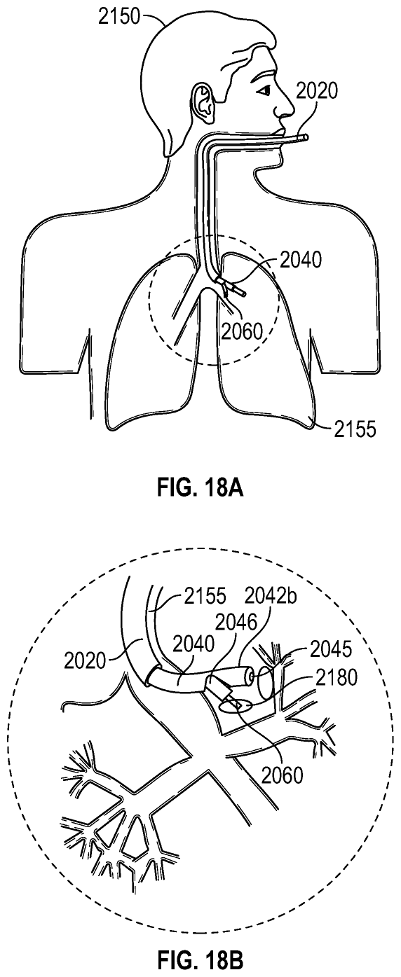

FIG. 18A illustrates a bronchoscope assembly 2010 including a bronchoscope 2020 and a catheter assembly 2030 with a catheter 2040 and a surgical instrument 2060 inserted into the lungs 2155 via a natural orifice (e.g., the mouth) of a patient 2150 toward the target following a pathway plan. When the bronchoscope 2020 reaches a certain location of the lung 2155, the bronchoscope 2020 becomes wedged and cannot go further into bronchial tree due to the size constraints. Then, the catheter 2040 including an EWC 2044 may be used to navigate the luminal network to a target 2180 following the pathway plan, as described above. The EWC 2044 is small and thin enough to reach the target 2180.

FIG. 18B illustrates an enlarged detail view of the circled area of FIG. 18A, where the catheter 2040, and particularly the distal end portion 2042b, exited a natural lumen of the lung 2155 to butt up against the target tissue 2180. The surgical instrument 2060 is depicted extending from the angled side catheter port 2046 of the catheter 2040 between the US transducer 2190 and the target tissue 2180. In this configuration, the treatment or biopsy of the target tissue can be performed with US imaging to ensure proper placement of the assemblies and/or surgical instruments described herein. Although not depicted, in some embodiments, the surgical instrument may further or alternatively be rotated to extend from the distal catheter port to the same or a different target tissue.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting but merely as exemplifications of particular embodiments. Those skilled in the art will envision other modifications within the scope and spirit of the claims appended hereto.

What is claimed is:

1. An endoscope assembly for navigation within a luminal structure comprising:
an endoscope including a shaft having an endoscope sidewall defining an endoscope channel therein, a distal endoscope port positioned on a distal end of the shaft and in communication with a distal end of the endoscope channel, and an angled side endoscope port defined through a distal portion of the endoscope sidewall and in communication with the endoscope channel, and
a catheter configured for positioning in the endoscope channel to extend distally through the distal endoscope port or extend laterally through the angled side endoscope port.

2. The endoscope assembly of claim 1, wherein the catheter is configured to be curved.

3. The endoscope assembly of claim 2, wherein the catheter is pre-curved.

4. The endoscope assembly of claim 2, wherein the catheter is steerable.

5. The endoscope assembly of claim 1, wherein the angled side endoscope port includes a compound opening.

6. The endoscope assembly of claim 5, wherein the compound opening defines at least a first edge along the endoscope sidewall and extending generally perpendicular to a longitudinal axis of the endoscope channel and a second edge along the endoscope sidewall and extending at an acute angle relative to the longitudinal axis of the endoscope channel.

7. The endoscope assembly of claim 5, wherein the compound opening includes at least a first and second edges along the endoscope sidewall, wherein the first and second edge form a first angle therebetween ranging from about 25° to about 85°.

8. The endoscope assembly of claim 7, wherein the first angle ranges from about 35° to about 75°.

9. The endoscope assembly of claim 1, further comprising one or more ultrasound transducers.

10. The endoscope assembly of claim 1, wherein the endoscope is a bronchoscope.

11. The endoscope assembly of claim 1, further comprising one or more surgical instruments configured for positioning in a working channel of the catheter to extend distally through a distal catheter port.

12. The endoscope assembly of claim 11, wherein the one or more surgical instrument is selected from the group consisting of a locating guide, imaging device, guidewire, surgical balloon, biopsy forceps, cytology brush, aspirating needle, ablation device, and combinations thereof.

13. A catheter assembly for navigation within a luminal structure comprising:
a catheter including a tube having a catheter sidewall defining a working channel therein, a distal catheter port positioned on a distal end of the tube and in communication with the working channel, and an angled side catheter port defined through a distal portion of the catheter sidewall and in communication with the working channel, and
a surgical instrument configured for positioning in the working channel to extend distally through the distal catheter port or extend laterally through the angled side catheter port.

14. The catheter assembly of claim 13, wherein the surgical instrument is configured to be curved.

15. The catheter assembly of claim 14, wherein the surgical instrument is pre-curved or steerable.

16. The catheter assembly of claim 13, wherein the angled side catheter port includes a compound opening defining at least a first edge along the catheter sidewall and extending generally perpendicular to a longitudinal axis of the working channel and a second edge along the catheter sidewall and extending at an acute angle relative to the longitudinal axis of the king channel.

17. The catheter assembly of claim 18, wherein the compound opening includes at least a first and second edges along the catheter sidewall, wherein the first and second edge forming a first angle therebetween ranging from about 25° to about 85°.

18. The catheter assembly of claim 13, further comprising an ultrasound transducer.

19. The catheter assembly of claim 13, wherein the surgical instrument is selected from the group consisting of a locating guide, imaging device, guidewire, surgical balloon, biopsy forceps, cytology brush, aspirating needle, ablation device, and combinations thereof.

20. An electromagnetic navigation system for navigating through a luminal network of a patient's lung, the system comprising:
a computing device,
a monitoring device,
an electromagnetic board,
a tracking device, and
a bronchoscope assembly including at least one of a bronchoscope including a distal bronchoscope port and an angled side bronchoscope port or a catheter including a distal catheter port and an angled side catheter port, and optionally a surgical instrument.
